(19) United States
(12) Patent Application Publication
(10) Pub. No.: US 2024/0341758 A1
(43) Pub. Date: Oct. 17, 2024

(54) HANDHELD ELECTROMECHANICAL SURGICAL SYSTEM

(71) Applicant: Covidien LP, Mansfield, MA (US)

(72) Inventors: Christopher K. Evans, Southington, CT (US); Robert H. Knapp, Middlebury, CT (US)

(21) Appl. No.: 18/751,692

(22) Filed: Jun. 24, 2024

Related U.S. Application Data
(63) Continuation of application No. 17/246,828, filed on May 3, 2021, now Pat. No. 12,016,556.

(51) Int. Cl. A61B 17/068; A61B 17/072; A61B 90/00

(52) U.S. Cl. CPC A61B 17/0686; A61B 17/072; A61B 90/06; A61B 2017/07257; A61B 2017/07271; A61B 2017/07278; A61B 2017/07285; A61B 2090/064

(57) ABSTRACT

A surgical device includes an end effector having a pair of opposing jaw members and a drive beam movable longitudinally through the pair of opposing jaw members thereby approximating the pair of opposing jaw members relative to each other. The device also includes an adapter assembly configured to selectively couple to the end effector. The adapter assembly includes an actuation assembly configured to mechanically engage the drive beam and to move the drive beam longitudinally. The device also includes a handle assembly configured to selectively couple to the adapter assembly. The handle assembly includes: a power source, a motor coupled to the power source, a sensor configured to measure a force imparted on the drive beam, and a motor controller configured to control the motor to maintain constant force on the drive beam based on the force measured by the sensor during longitudinal movement of the drive beam approximating the pair of opposing jaw members closer relative to each other.

### CROSS-REFERENCE TO RELATED APPLICATIONS

This application is a continuation application of U.S. patent application Ser. No. 17/246,828 filed on May 3, 2021, now U.S. Pat. No. 12,016,556. The entire contents of the foregoing application are incorporated by reference herein.

### BACKGROUND

### 1. Technical Field

The present disclosure relates to surgical devices. More specifically, the present disclosure relates to handheld electromechanical surgical systems for performing surgical procedures.

### 2. Background of Related Art

Surgical fastener devices for applying fasteners or staples to tissue are well known. These fastener devices include single-use devices which are preloaded with one or more staples and are disposable after a single use. Multiple use devices are also available and are preloaded with a plurality of staples. Multiple use devices may include a handle assembly that is electromechanically, e.g., powered, or manually actuated. These devices may be used with single use loading units (SULU) or multiple use loading units (MULU). The loading units include a body and an end effector, and are attached to the handle assembly, either directly or via an adapter assembly couplable to the handle assembly. The loading units may also include an articulating end effector. In powered surgical devices, which utilize motors to actuate and/or articulate the end effector, the motors may result in slower ejection of staples than manually actuated surgical devices. Thus, there is a need for powered surgical staplers configured to eject staplers at a faster rate commensurate with manual staplers.

### SUMMARY

According to one embodiment of the present disclosure, a surgical device is disclosed. The surgical device includes an end effector having a pair of opposing jaw members and a drive beam movable longitudinally through the pair of opposing jaw members thereby approximating the pair of opposing jaw members relative to each other. The device also includes an adapter assembly configured to selectively couple to the end effector. The adapter assembly includes an actuation assembly configured to mechanically engage the drive beam and to move the drive beam longitudinally. The device also includes a handle assembly configured to selectively couple to the adapter assembly. The handle assembly includes: a power source, a motor coupled to the power source, a sensor configured to measure a force imparted on the drive beam, and a motor controller configured to control the motor to maintain constant force on the drive beam based on the force measured by the sensor during longitudinal movement of the drive beam approximating the pair of opposing jaw members closer relative to each other.

Implementations may include one or more of the following features. According to one aspect of the above embodiment, one jaw member of the pair of opposing jaw members may include a plurality of staples. The end effector may further include an actuation sled movable through the one jaw member of the pair of opposing jaw members to eject the plurality of staples. The drive beam may be configured to engage the actuation sled to move the actuation sled through the one jaw member of the pair of opposing jaw members. The drive beam may further include a distally facing knife. The motor controller includes a proportional-integral-derivative (PID) controller configured to receive the measured force as input and to output motor control signals to maintain the constant force on the drive beam. The motor control signals may be pulse-width-modulated. The motor controller may be further configured to ramp up to the constant force at a constant rate. The handle assembly may further include a user input button and a main controller configured to receive an input signal from the user input button and to signal the motor controller to control the motor.

According to another embodiment of the present disclosure, a surgical device is disclosed. The surgical device includes an end effector having an anvil assembly, a cartridge assembly having a plurality of staples, and a drive beam movable longitudinally through the anvil assembly and the cartridge assembly thereby approximating the anvil assembly and the cartridge assembly relative to each other and ejecting the plurality of staples. The surgical device also includes an adapter assembly configured to selectively couple to the end effector. The adapter assembly includes an actuation assembly configured to mechanically engage the drive beam and to move the drive beam longitudinally. The surgical device also includes a handle assembly configured to selectively couple to the adapter assembly. The handle assembly includes: a power source, a motor coupled to the power source, a sensor configured to measure a force imparted on the drive beam, and a motor controller configured to control the motor to maintain constant force on the drive beam based on the force measured by the sensor during movement of the drive beam to approximate the anvil assembly and the cartridge assembly relative to each other and eject the plurality of staples.

Implementations may include one or more of the following features. According to one aspect of the above embodiment, the end effector may include an actuation sled movable through the cartridge assembly and configured to eject the plurality of staples. The drive beam may be configured to engage the actuation sled to move the actuation sled through the cartridge assembly. The drive beam may further include a distally facing knife. The motor controller may include a proportional-integral-derivative (PID) controller configured to receive the measured force as input and to output motor control signals to maintain the constant force on the drive beam. The motor control signals may be pulse-width-modulated. The motor controller may be further configured to ramp up to the constant force at a constant rate.

According to a further embodiment of the present disclosure, a method for controlling a surgical device is disclosed. The method includes activating a motor coupled to a drive beam configured to move longitudinally through an anvil assembly and a cartridge assembly of an end effector. The method also includes approximating the anvil assembly and the cartridge assembly closer relative to each other. The method also includes ejecting a plurality of staples disposed in the cartridge assembly. The method further includes measuring a force imparted on the drive beam. The method further includes controlling the force imparted on the drive beam to remain constant at a setpoint during longitudinal movement.

Implementations may include one or more of the following features. According to one aspect of the above embodiment, the method may also include ramping up the force imparted on the drive beam at a constant rate to reach the setpoint. The method may further include advancing a knife through the anvil assembly and the cartridge assembly. The method may also include controlling the force by processing the measured force as input through a proportional-integral-derivative (PID) controller and outputting the motor control signals from the PID controller to maintain the constant force on the drive beam.

### BRIEF DESCRIPTION OF THE DRAWINGS

Embodiments of the present disclosure are described herein with reference to the accompanying drawings, wherein:

FIG. 1 is a perspective view of a handheld surgical device including a handle assembly, an adapter assembly, and a surgical loading unit according to an embodiment of the present disclosure;

FIG. 2 is a perspective view illustrating a connection of the adapter assembly and the handle assembly of FIG. 1 according to an embodiment of the present disclosure;

FIG. 3 is perspective view of internal components of the handle assembly according to an embodiment of the present disclosure;

FIG. 4 is cross-sectional view of the adapter assembly taken along a section plane “4-4” of FIG. 1 according to an embodiment of the present disclosure;

FIG. 5 is cross-sectional view of the adapter assembly taken along a section plane “5-5” of FIG. 1 according to an embodiment of the present disclosure;

FIG. 6 is a perspective view, with parts separated, of the adapter assembly of FIG. 1 according to an embodiment of the present disclosure;

FIG. 7 is a perspective view, with parts separated, of the surgical loading unit of FIG. 1 according to an embodiment of the present disclosure;

FIG. 8 is a schematic diagram of the handle assembly, the adapter assembly, and the end effector of FIG. 1;

FIG. 9 is flow chart of a control algorithm controlling the surgical instrument of FIG. 1 according to an embodiment of the present disclosure; and

FIG. 10 shows plots of a load imparted on a drive beam and the rotation speed of the motor during the control algorithm of FIG. 9 according to an embodiment of the present disclosure.

### DETAILED DESCRIPTION OF EMBODIMENTS

Embodiments of the presently disclosed surgical devices, and adapter assemblies for surgical devices and/or handle assemblies are described in detail with reference to the drawings, in which like reference numerals designate identical or corresponding elements in each of the several views. As used herein the term “distal” refers to that portion of the surgical instrument, or component thereof, farther from the user, while the term “proximal” refers to that portion of the surgical instrument, or component thereof, closer to the user.

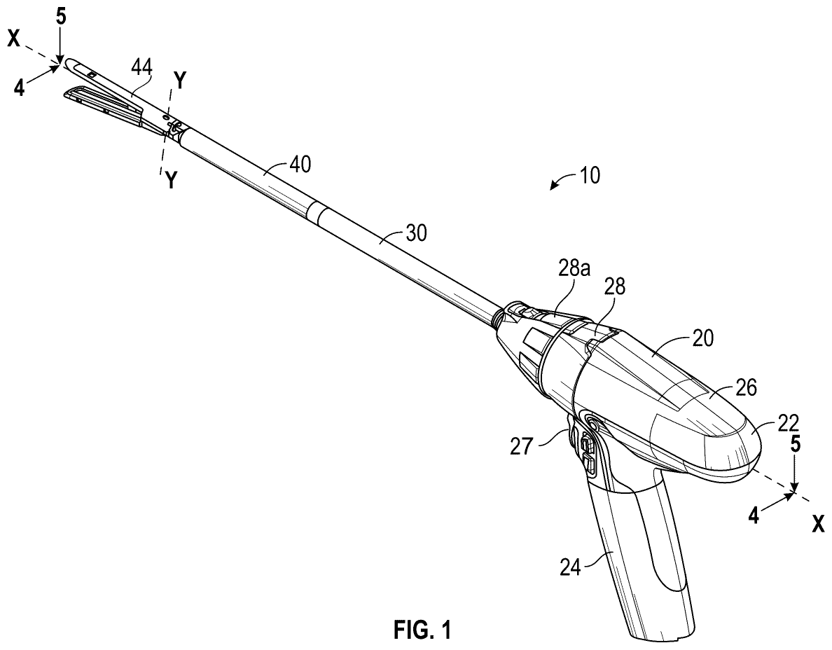

The present disclosure provides a powered surgical device 10 (e.g., stapler) having a handle assembly, an adapter assembly coupled to the handle assembly, and an end effector coupled to the adapter assembly. With reference to FIG. 1, a powered surgical device 10 includes a handle assembly 20, which is configured for selective connection with an adapter assembly 30, which in turn, is configured for selective connection with a loading unit 40 having an end effector 44. Although generally referred to as being a powered surgical device, it is contemplated that the surgical device 10 may be manually actuated and may include various configurations.

The handle assembly 20 includes a handle housing 22 having a lower housing portion 24, an intermediate housing portion 26 extending from and/or supported on a portion of the lower housing portion 24, and an upper housing portion 28 extending from and/or supported on a portion of the intermediate housing portion 26. The handle assembly 20 also includes a plurality of controls 23, which may include buttons 27, touchscreen, or any other user input devices, for allowing the clinician to control the operation of the surgical device 10.

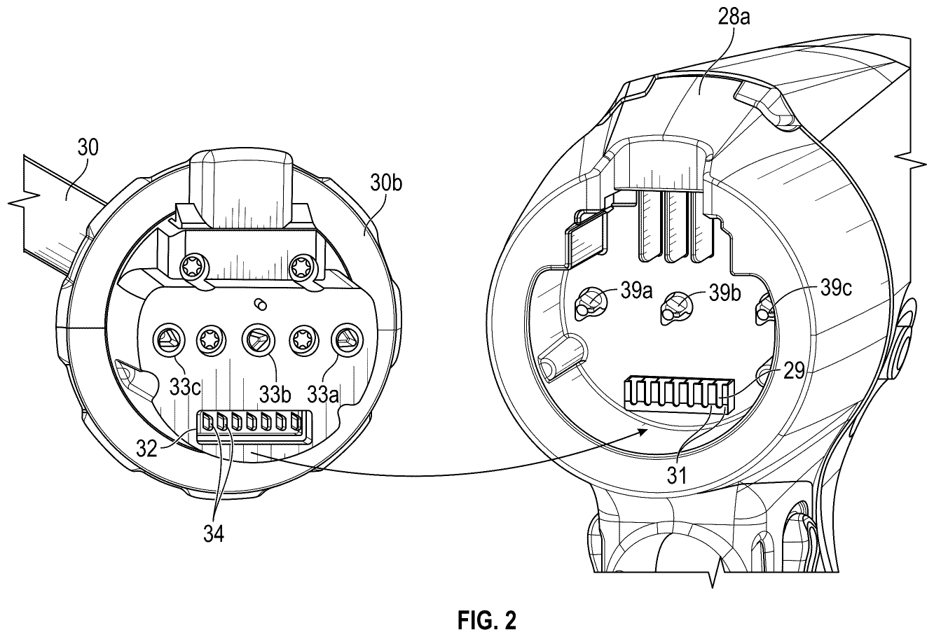

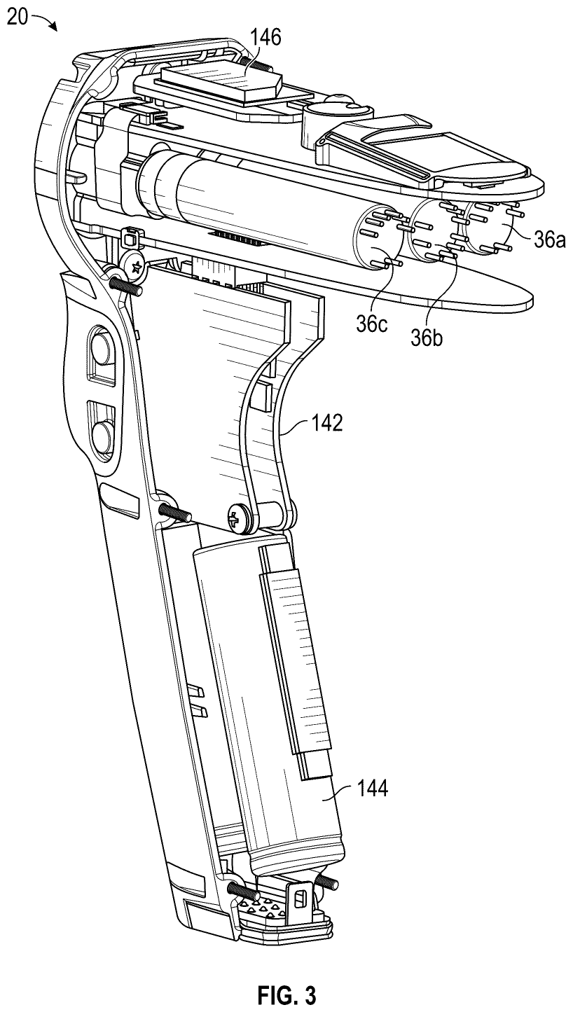

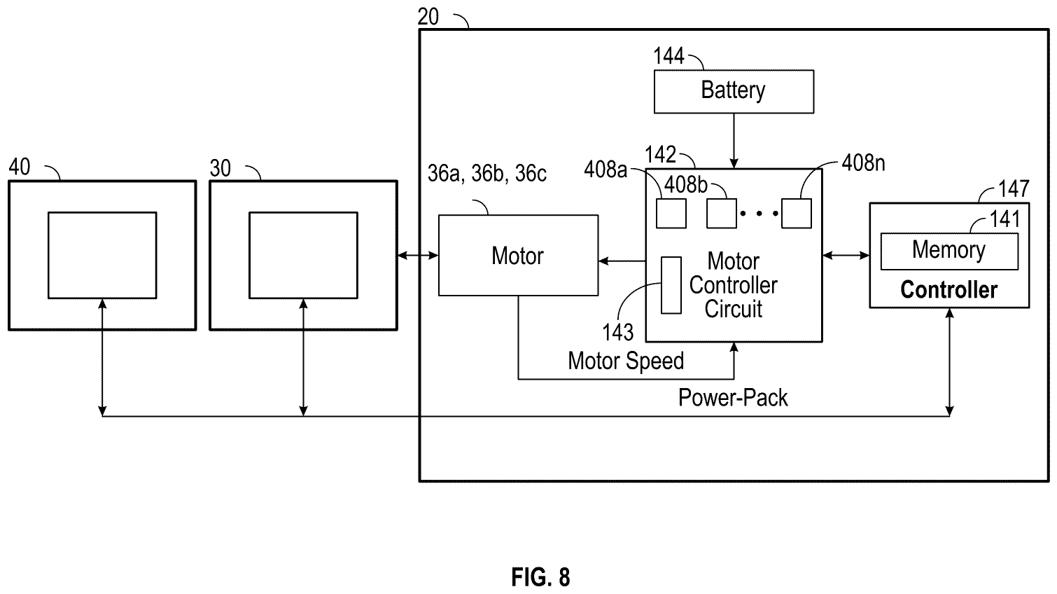

As shown in FIG. 2, a distal portion of the upper housing portion 28 defines a nose or connecting portion 28a that is configured to accept a proximal end portion 30b of the adapter assembly 30. The connecting portion 28a of the upper housing portion 28 includes an electrical receptacle 29 having a plurality of electrical contacts 31, which are in electrical communication with electronic (e.g., a main controller circuit board 142 of FIG. 8) and electrical components (e.g., a rechargeable battery 144 of FIGS. 3 and 8) of the handle assembly 20. The adapter assembly 30 includes a counterpart electrical connector 32 that is configured to engage the electrical receptacle 29. The electrical connector 32 also includes a plurality of electrical contacts 34 that engage and electrically connect to their counterpart electrical contacts 31.

With reference to FIG. 3, the handle assembly 20 includes motors 36a, 36b, 36c, which are coupled to corresponding drive shafts 39a, 39b, 39c (FIG. 2), which are configured to engage respective sockets 33a, 33b, 33c on the proximal end portion 30b, such that rotation of the drive shafts 39a, 39b, 39c is imparted on the sockets 33a, 33b, 33c.

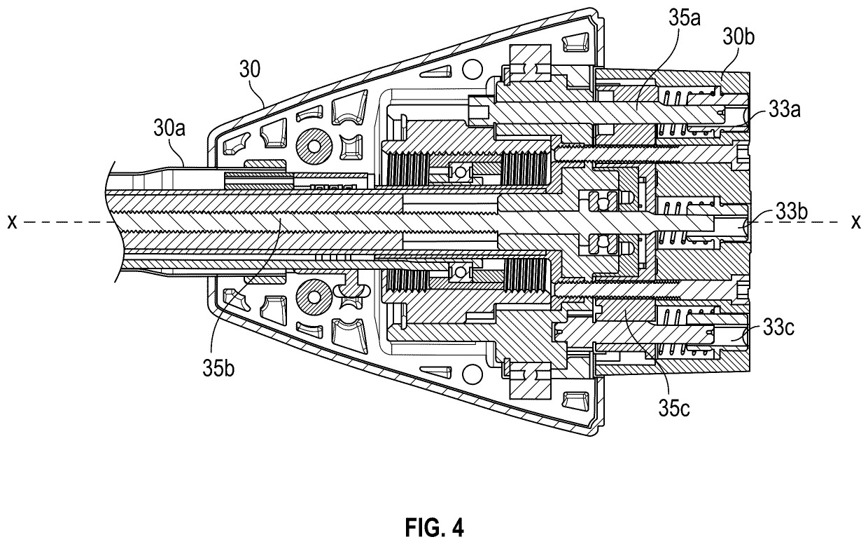

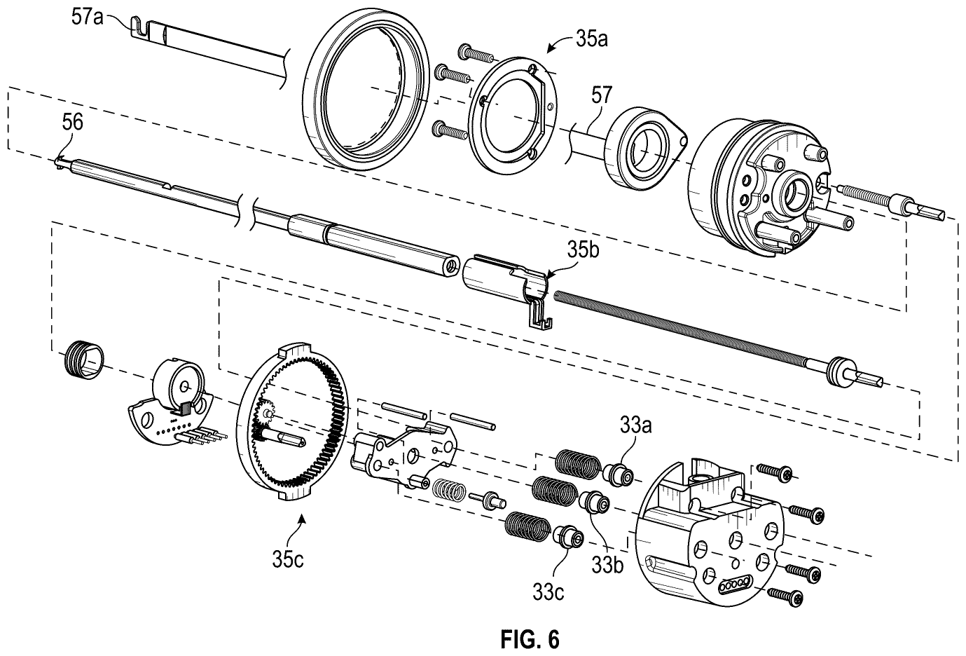

With reference to FIGS. 4-6, the adapter assembly 30 includes a tubular housing 30a that extends between a proximal end portion 30b that is configured for operable connection to the connecting portion 28a of the handle assembly 20 and an opposite, distal end portion 30c that is configured for operable connection to the loading unit 40. The adapter assembly 30 includes actuation assemblies 35a, 35b, 35c each of which is coupled to one of the sockets 33a, 33b, 33c (FIG. 6). The actuation assemblies 35a, 35b, 35c are configured to transfer rotational motion of the sockets 33a, 33b, 33c into linear motion and/or rotational motion, such that the adapter assembly 30 is configured to convert a rotational motion provided by the handle assembly 20 into axial translation for rotating the adapter assembly 30 about a longitudinal axis X-X, articulate the loading unit 40, clamp tissue, eject fasteners, and cut fastened tissue.

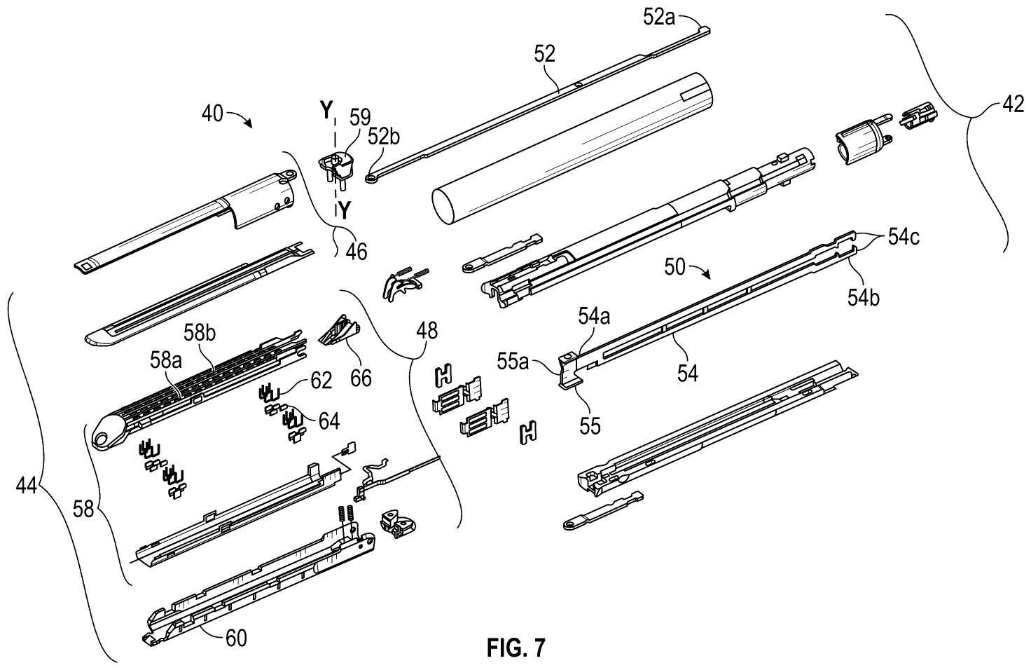

With reference to FIG. 6, the actuation assembly 35a, actuated by the motor 36a, includes an articulation link 57 having a hook 57a disposed at a distal end portion of the articulation link 57. Longitudinal movement of the articulation link 57 is used to articulate the end effector 44 about an articulation axis Y-Y (FIGS. 1 and 7), which is perpendicular to the longitudinal axis X-X. The actuation assembly 35b, actuated by the motor 36b, includes a drive member 56, which is used to operate an anvil assembly 46 and a cartridge assembly 48 of the end effector 44. The actuation assembly 35c, actuated by the motor 36c, is used to rotate the adapter assembly 30 about the longitudinal axis X-X.

With reference to FIGS. 1 and 7, an embodiment of the loading unit 40 is shown. The loading unit 40 includes a proximal body portion 42 and the end effector 44. Proximal body portion 42 is releasably attached to the distal end portion 30c of adapter assembly 30, and end effector 44 is pivotally attached to a distal end of proximal body portion 42. End effector 44 includes the anvil assembly 46 and the cartridge assembly 48. Anvil assembly 46 is pivotable in relation to the cartridge assembly 48 and is movable between an open or unclamped position and a closed or clamped position. Proximal body portion 42 includes a drive assembly 50 and an articulation link 52.

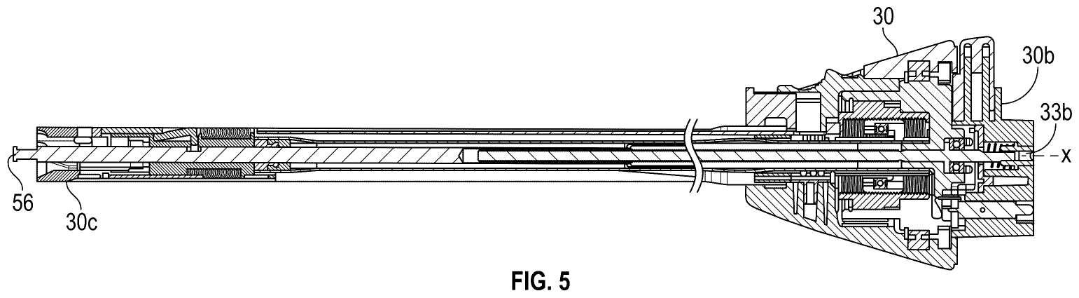

Drive assembly 50 includes a flexible drive beam 54 having a distal end portion 54a and a proximal engagement section 54b. The distal end portion 54a includes an I-beam 55 having a knife 55a. The I-beam 55 is configured to travel through the anvil assembly 46 and the cartridge assembly 48, thereby pushing the anvil assembly 46 toward the cartridge assembly 48 to clamp tissue. The proximal engagement section 54b includes diametrically opposed inwardly extending fingers 54c that engage the drive member 56 (FIGS. 5 and 6) to fixedly secure drive member 56 to the proximal end of flexible drive beam 54. Drive member 56 is actuated by the actuation assembly 35b of adapter assembly 30.

Cartridge assembly 48 of end effector 44 includes a staple cartridge 58 removably supported in a carrier 60. Staple cartridge 58 defines a central longitudinal slot 58a, and a plurality of linear rows of staple retention slots 58b positioned on each side of the central longitudinal slot 58a. Each of the staple retention slots 58b receives a staple 62 and a portion of a staple pusher 64. During operation of the surgical device 10, drive assembly 50 abuts an actuation sled 66 and pushes actuation sled 66 through the staple cartridge 58. As the actuation sled 66 moves through staple cartridge 58, cam wedges of the actuation sled 66 sequentially engage staple pushers 64 to move staple pushers 64 vertically within staple retention slots 58b and sequentially eject the staples 62 therefrom for formation against an anvil plate 46a of anvil assembly 46.

Proximal body portion 42 of loading unit 40 includes an articulation link 52 having a hooked proximal end portion 52a which extends from a proximal end of loading unit 40 which engages the opposing articulation link 57 coupled to the actuation assembly 35a of the adapter assembly 30. Articulation link 52 has a distal end portion 52b pivotably secured to end effector 44. As the articulation link 57 is moved in an axial direction by the actuation assembly 35a of the adapter assembly 30, either proximally or distally, the articulation link 52 of the loading unit 40 is also moved in the same manner. Axial movement of the articulation link 52, in turn, articulates (e.g., pivots) the end effector 44 about a pivot pin 59, which defines the axis Y-Y.

With reference to FIG. 8, the handle assembly 20 includes the main controller circuit board 142, the rechargeable battery 144 configured to supply power to any of the electrical components of handle assembly 100, and a plurality of motors 36a, 36b, 36c coupled to the battery 144. The handle assembly 20 also includes the display 146. In embodiments, the motors 36a, 36b, 36c may be coupled to any suitable power source configured to provide electrical energy to the motors 36a, 36b, 36c, such as an AC/DC transformer. Each of the motors 36a, 36b, 36c is coupled a motor controller 143 which controls the operation the motors 36a, 36b, 36c, including the flow of electrical energy from the battery 144 to the motors 36a, 36b, 36c. A main controller 147 is provided that controls the handle assembly 20. The main controller 147 is configured to execute software instructions embodying algorithms disclosed herein, such as clamping, stapling, and cutting algorithms which control operation of the handle assembly 20.

The motor controller 143 includes a plurality of sensors 408 (e.g., 408a . . . 408n) configured to measure operational states of the motors 36a, 36b, 36c and the battery 144. The sensors 408 include a strain gauge 408b and may also include voltage sensors, current sensors, temperature sensors, telemetry sensors, optical sensors, and combinations thereof. The sensors 408 may measure voltage, current, and other electrical properties of the electrical energy supplied by the battery 144. One of the sensors 408, namely, a sensor 408a may measure angular velocity (e.g., rotational speed) as revolutions per minute (RPM), torque, temperature, current draw, and other operational properties of the motor 36a. The sensor 408a may also include an encoder configured to count revolutions or other indicators of the motor 36a, which is then use by the main controller 147 to calculate linear movement of components movable by the motor 36a. Angular velocity may be determined by measuring the rotation of the motors 36a, 36b, 36c or a drive shaft (not shown) coupled thereto and rotatable by the motors 36a, 36b, 36c. The position of various axially movable drive shafts may also be determined by using various linear sensors disposed in or in proximity to the shafts or extrapolated from the RPM measurements. In embodiments, torque may be calculated based on the regulated current draw of the motors 36a, 36b, 36c at a constant RPM. In further embodiments, the motor controller 143 and/or the main controller 147 may measure time and process the above-described values as a function of time, including integration and/or differentiation, e.g., to determine the rate of change in the measured values. The main controller 147 is also configured to determine distance traveled of various components of the adapter assembly 200 and/or the end effector 300 by counting revolutions of the motor 36a.

The motor controller 143 is coupled to the main controller 147, which includes a plurality of inputs and outputs for interfacing with the motor controller 143. In particular, the main controller 147 receives measured sensor signals from the motor controller 143 regarding operational status of the motors 36a, 36b, 36c and the battery 144 and, in turn, outputs control signals to the motor controller 143 to control the operation of the motors 36a, 36b, 36c based on the sensor readings and specific algorithm instructions. The main controller 147 is also configured to accept a plurality of user inputs from a user interface (e.g., switches, buttons, touch screen, etc. coupled to the main controller 147).

The main controller 147 is also coupled to a memory 141. The memory 141 may include volatile (e.g., RAM) and non-volatile storage configured to store data, including software instructions for operating the handle assembly 20. The main controller 147 is also coupled to the strain gauge 408b of the adapter assembly 200 using a wired or a wireless connection and is configured to receive strain measurements from the strain gauge 408b which are used during operation of the handle assembly 20.

The handle assembly 20 includes a plurality of motors 36a, 36b, 36c each including a respective motor shaft (not explicitly shown) extending therefrom and configured to drive a respective transmission assembly. Rotation of the motor shafts by the respective motors function to drive shafts and/or gear components of adapter assembly 200 in order to perform the various operations of handle assembly 100.

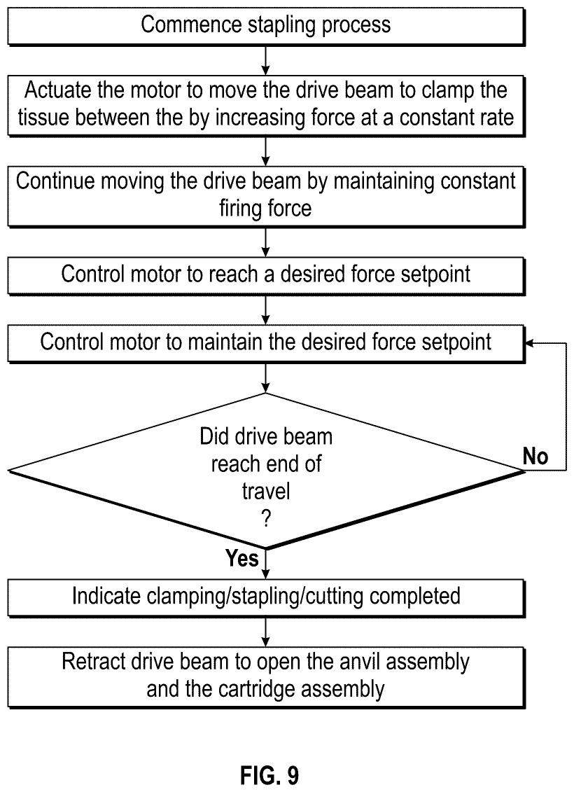

A method for actuating the end effector 44 to clamp, staple, and cut the tissue is shown in FIG. 9. The process is commenced by positioning the tissue between the anvil assembly 46 and the cartridge assembly 48 and pressing one of the buttons 27. The main controller 147 receives the input from one of the buttons 27 and instructs the motor controller 143 to output a control signal to the motor 36b responsible for actuating the end effector 44. The motor controller 143 is configured to output a pulse-width modulated (PWM) control signal, which allows to adjust the power supplied to the motor 36b in discrete increments. The motor controller 143 may include a proportional-integral-derivative (PID) controller or any other suitable control loop circuit using feedback signals to achieve a desired setpoint. The parameters of the PID loop or control loop can be configurable based on various inputs, data collected during clamp, reload type, tissue type e.g., sensing tissue type or manually entering tissue type.

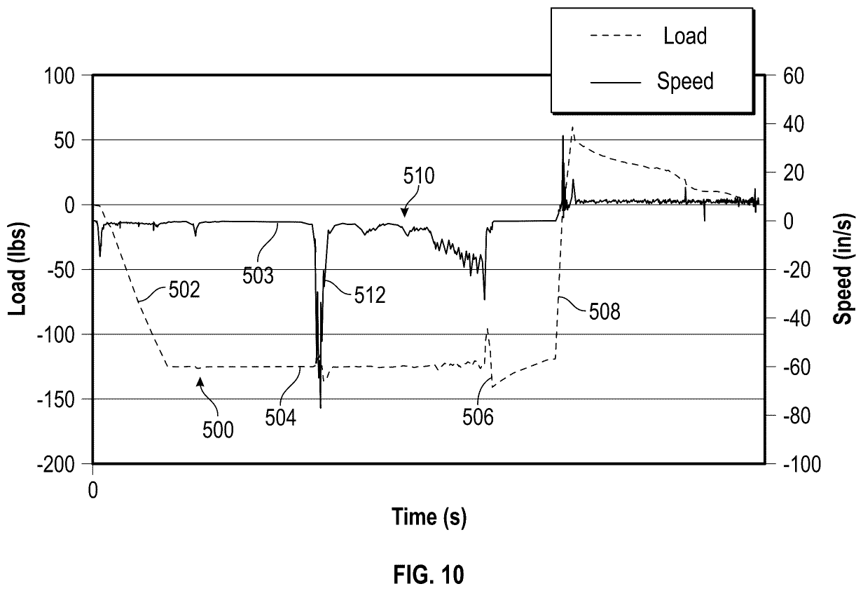

The motor controller 143 controls the motor 36b to move the drive beam 54 in a distal direction to commence clamping. In response, the drive beam 54 closes the anvil assembly 46 and the cartridge assembly 48 and simultaneously advances the knife 55a and the actuation sled 66. As a result of actuation of these components a force is imparted on the drive beam 54. FIG. 10 which shows superimposed plots 500 and 510, with the plot 500 showing the load imparted on the drive beam 54 as measured by the strain gauge 408b and the plot 510 illustrating the rotation speed of the motor 36b and/or linear speed of the drive beam 54, respectively.

Initially, the motor controller 143 adjusts the motor 36b to ramp the force at a constant rate as shown in a segment 502 of the force plot 500 until a setpoint force is reached. Once the setpoint is reached, the force is maintained during a segment 504. The speed of the motor 36b may vary during the constant force segment 504 as shown by a spike 512 in the speed plot 510, while the constant force is maintained. In embodiments, the linear speed of the drive beam 54 and/or the rotation speed of the motor 36b may also be held constant during a segment 503, at least until the spike 512 is reached, which is contemporaneous with the constant force segment 504. The drive beam 54 is advanced while the constant force is maintained during the segment 504 until the drive beam 54 reaches an end of travel position, which corresponds to the knife 55a and/or the anvil assembly 46.

End of travel may be determined using the sensor 408a, which determines position of the drive beam 54 based on the revolutions of the motor 36b or any other suitable position sensor. In embodiments, end of travel may be determined using the strain gauge 408b which can detect a mechanical stop encountered by the drive beam 54. Once end of travel is determined, the motor 36b stops advancing the drive beam 54 and withdraws the drive beam 54 to open the anvil assembly 46 and the cartridge assembly 48. End of travel and withdrawal of the drive beam 54 is indicated by a spike 506 and a segment 508, respectively. More specifically, the spike 506 indicates reaching the mechanical limit and the segment 508 indicates reversal of the drive beam 54.

It is envisioned that the constant force algorithm according to the present disclosure may be used with any jaw type powered or robotic surgical instrument as well as end-to-end anastomosis circular staplers, and the like. It will be understood that various modifications may be made to the embodiments of the presently disclosed adapter assemblies. Therefore, the above description should not be construed as limiting, but merely as exemplifications of embodiments. Those skilled in the art will envision other modifications within the scope and spirit of the present disclosure.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A surgical device comprising:
a pair of opposing jaw members; and
a drive beam movable longitudinally through the pair of opposing jaw members thereby approximating the pair of opposing jaw members relative to each other;
a motor configured to move the drive beam;
a sensor configured to measure a force imparted on the drive beam; and
a motor controller configured to control the motor to maintain constant force on the drive beam based on the force measured by the sensor during longitudinal movement of the drive beam approximating the pair of opposing jaw members relative to each other.

2. The surgical device according to claim 1, wherein one jaw member of the pair of opposing jaw members includes a plurality of staples.

3. The surgical device according to claim 2, further including an actuation sled movable through the one jaw member of the pair of opposing jaw members to eject the plurality of staples.

4. The surgical device according to claim 3, wherein the drive beam is configured to engage the actuation sled to move the actuation sled through the one jaw member of the pair of opposing jaw members.

5. The surgical device according to claim 1, wherein the drive beam further includes a distally facing knife.

6. The surgical device according to claim 1, wherein the motor controller includes a proportional-integral-derivative (PID) controller configured to receive the measured force as input and to output motor control signals to maintain the constant force on the drive beam.

7. The surgical device according to claim 6, wherein the motor control signals are pulse-width-modulated.

8. The surgical device according to claim 6, wherein the motor controller is further configured to ramp up to the constant force at a constant rate.

9. The surgical device according to claim 1, further comprising:
a handle assembly including:
a user input button; and
a main controller configured to receive an input signal from the user input button and to signal the motor controller to control the motor.

10. A surgical device comprising:
an end effector including:
an anvil assembly;
a cartridge assembly having a plurality of staples; and
a drive beam movable longitudinally through the anvil assembly and the cartridge assembly thereby approximating the anvil assembly and the cartridge assembly relative to each other and ejecting the plurality of staples;
an adapter assembly configured to selectively couple to the end effector, the adapter assembly including an actuation assembly configured to mechanically engage the drive beam and to move the drive beam longitudinally; and
a handle assembly configured to selectively couple to the adapter assembly, the handle assembly including:
a power source;
a motor coupled to the power source;
a sensor configured to measure a force imparted on the drive beam; and
a motor controller configured to control the motor to maintain constant force on the drive beam based on the force measured by the sensor during longitudinal movement of the drive beam to approximate the anvil assembly and the cartridge assembly relative to each other and eject the plurality of staples.

11. The surgical device according to claim 10, wherein the end effector further includes an actuation sled movable through the cartridge assembly and configured to eject the plurality of staples.

12. The surgical device according to claim 11, wherein the drive beam is configured to engage the actuation sled to move the actuation sled through the cartridge assembly.

13. The surgical device according to claim 10, wherein the drive beam further includes a distally facing knife.

14. The surgical device according to claim 10, wherein the motor controller includes a proportional-integral-derivative (PID) controller configured to receive the measured force as input and to output motor control signals to maintain the constant force on the drive beam.

15. The surgical device according to claim 14, wherein the motor control signals are pulse-width-modulated.

16. The surgical device according to claim 14, wherein the motor controller is configured to ramp up to the constant force at a constant rate.

17. A method for controlling a surgical device, the method comprising:
activating a motor coupled to a drive beam configured to move longitudinally through an anvil assembly and a cartridge assembly of an end effector;
approximating the anvil assembly and the cartridge assembly relative to each other;
ejecting a plurality of staples disposed in the cartridge assembly;
measuring force imparted on the drive beam; and
controlling the force imparted on the drive beam to remain constant at a setpoint during longitudinal movement.

18. The method according to claim 17, further comprising ramping up the force imparted on the drive beam at a constant rate to reach the setpoint.

19. The method according to claim 17, further comprising:
advancing a knife through the anvil assembly and the cartridge assembly.

20. The method according to claim 17, wherein controlling the force further includes:
processing the measured force as input at a proportional-integral-derivative (PID) controller; and
outputting motor control signals from the PID controller to maintain the constant force on the drive beam.
